(19) United States
(12) Patent Application Publication
(10) Pub. No.: US 2013/0165334 A1
(43) Pub. Date: Jun. 27, 2013

(54) INTEGRATED ASSAY THAT COMBINES FLOW-CYTOMETRY AND MULTIPLEXED HPV GENOTYPE IDENTIFICATION

(71) Applicants: Andrew A. Bieberich, West Lafayette, IN (US); Lova N. Rakotomalala, West Lafayette, IN (US); Vincent J. Davisson, West Lafayette, IN (US); Joseph P. Robinson, West Lafayette, IN (US)

(72) Inventors: Andrew A. Bieberich, West Lafayette, IN (US); Lova N. Rakotomalala, West Lafayette, IN (US); Vincent J. Davisson, West Lafayette, IN (US); Joseph P. Robinson, West Lafayette, IN (US)

(21) Appl. No.: 13/582,558

(22) PCT Filed: Mar. 4, 2011
(86) PCT No.: PCT/US11/27181

§ 371 Date: Mar. 5, 2013

Related U.S. Application Data
(60) Provisional application No. 61/310,368, filed on Mar. 4, 2010.

(51) Int. Cl. C12Q 1/70

(52) U.S. Cl. CPC C12Q 1/708
USPC 506/9; 536/24.33

(57) ABSTRACT

A two part assay is disclosed that enables collection of both protein biomarker phenotype and specific HPV genotype data from within a clinically derived population of cervical epithelial cells. Presence of multiple transformation-associated protein biomarkers acts as a gating criterion for cell sorting, followed by application of a PCR protocol sensitive enough to detect and identify individual HPV types from within the cells captured during sorting. The workflow has been optimized to work with cells conventionally fixed in PreservCyt (Cytyc), and it can be performed on residual cells remaining in a stored sample after a Pap test has been performed.

### US GOVERNMENT RIGHTS

This disclosed assays and methods were invented with Government support under Contract No.: R21 CAl25370, awarded by the National Institute of Health (NIH). The U.S. Government may have certain rights to this application.

### BACKGROUND

1. Technical Field

A two part assay is disclosed that enables collection of both protein biomarker phenotype and specific HPV genotype data from within a clinically derived population of cervical epithelial cells.

2. Description of the Related Art

Cervical cancer is second to breast cancer as the most common form of malignancy in both incidence and mortality for women worldwide. The population-wide utilization of screening cervical cytology (Pap smear tests or “Pap tests”) has been associated with a dramatic decrease in morbidity and mortality from cervical cancer in the United States and in other industrialized nations. Despite the success of Pap tests, the cytological diagnosis of cervical lesions is plagued by a persistent problem of low specificity for clinically significant high-grade lesions in patients with low-grade cytological abnormalities. As a result, over 4 million women each year receive a cytological diagnosis that requires further evaluation to rule out the possibility of high-grade dysplasia or cancer. In most cases, further evaluation does not identify underlying high-grade lesions in patients with low-grade cytological abnormalities. Although a simple detection of high-risk types of human papillomavirus can play an important role for the triage of patients, the detection, by itself, has not been useful for several cytological diagnoses. Specifically, simple detection of high risk HPVs does not predict an underlying high grade lesion because of the long lag time (up to 10 years) between initial infection and development of disease. Further, some studies indicate that prognosis may vary even among separate high-risk HPV types, yet the current commercially available test for high-risk HPV does not specify individual HPV types.

Cytological diagnosis of premalignant lesions of the cervical mucosa includes premalignant lesions of the cervical mucosa, which are detected by cytological examination of the Papanicolaou preparation (Pap smear test). Cytological findings are classified by the Bethesda system as normal/benign reactive changes (Normal/BRC), squamous cell abnormalities, and glandular cell abnormalities. Squamous cell abnormalities include atypical squamous cells of undetermined significance (ASCUS), low grade squamous intraepithelial lesions (LSIL), encompassing evidence of human papillomavirus (HPV) infection and/or mild dysplasia, and high grade intraepithelial lesions (HSIL), including moderate dysplasia (CIN 2) and severe dysplasia/carcinoma in situ (CIN 3). In the revised Bethesda 2001 system, the general category of ASCUS was replaced with two new diagnostic categories: “atypical squamous cells—of undetermined significance” (ASC-US) and “atypical squamous cells—cannot exclude HSIL” (ASC-H). Cases that would previously be classified as ASCUS, favoring a reactive process, are included in the category of benign cellular changes in the new classification system. The classification of LSIL and HSIL remained relatively unchanged from the previous Bethesda classification system but an additional subcategory of HSIL was created to report cases that have features that are suspicious for invasive carcinoma. Potentially premalignant lesions of the endocervical glandular mucosa can also be recognized in cervical cytological specimens. In the revised 2001 Bethesda system of cytological classification, glandular cell abnormalities that are less severe than adenocarcinoma are categorized as atypical glandular cells (AGC), either endocervical, endometrial, or “glandular cells” not otherwise specified (AGC NOS); atypical glandular cells, either endocervical cells or “glandular cells” favor neoplasia (AGC “favor neoplasia”), and endocervical adenocarcinoma in situ (AIS).

The Papanicolaou test is an effective screening tool for cancer. However, despite its success, the practice of diagnostic cytopathology is limited by poor specificity for underlying clinically significant lesions in cases with low-grade cytological abnormalities. Over 4 million cases are diagnosed as ASC-US, ASC-H, LSIL, or AGC in the United States each year, requiring further evaluation to identify the subset of patients that will have clinically significant high-grade lesions found on cervical biopsy (Table 1). In most cases, however, further evaluation does not identify high-grade squamous or glandular lesions in patients with low-grade cytological abnormalities. Patients with a cytological diagnosis of ASC (not otherwise specified) have a 5 to 17% of underlying CIN 2/3 on cervical biopsy and the diagnosis of ASC-H denotes a 24 to 94% chance of CIN 2/3 on colposcopic biopsy. In LSIL cases that were referred for colposcopic examination, high grade cervical dysplasia (CIN grade 2 or 3) was found in 25%, CIN 1 was found in 45%, but no dysplasia was found in over 25% of LSIL cases. The cytological diagnosis of AGC is also equivocal for the presence of an underlying clinically significant lesion of the cervical mucosa. Cases with a diagnosis of AGC have been found to have a 9 to 41% risk of CIN2, CIN 3, or AIS but the majority of AGC cases do not have biopsy confirmation of clinically significant lesions of either the squamous or glandular mucosa.

TABLE 1
Clinically Equivocal Cytological Diagnostic Categories

| | | | No clinically | |
|---|---|---|---|---|
| Cytological | Total cases | | significant | |
| Diagnosis | (Annual US population)* | | lesion on colposcopy | |
| ASC-US | >2 | million | 1.66-1.9 | million |
| ASC-H | 0.20 | million (estimated) | 0.001-0.15 | million |
| LSIL | 1.65 | million | 1.24 | million |
| AGC | 0.31 | million | 0.18-0.25 | million |
| Total | >4.16 | million | 2.66-3.54 | million |

The above statistics regarding performance of the Pap test of course do not even address the problem of lack of implementation in developing nations, where mortality rate per 100,000 women is 3-7 fold that of more industrialized areas of the world. Another issue with current practice is that clinicians are under pressure to read large numbers of specimens quickly. Cytotechnologists may have a screening rate of approximately 4-5 minutes per slide, or 100 slides in 8 hours. In other words, within 4 or 5 minutes, a cytotechnologist needs to visualize between 50,000 to 300,000 cells on a slide and to identify as few as 10 to 50 dysplastic cells in a positive specimen. Among all screened specimens, approximately 90% are negative. Therefore, most of the screener's time and energy is expended looking at healthy cells. Fatigue and monotony can reduce the acuity of the screener so that rare positive cells have a greater chance of being overlooked.

Cervical cancer detection at the molecular level includes detecting HPV oncogenes E6 and E7, and detecting p16^{IN4a} overexpression. Specifically, human papillomavirus has been categorized into approximately 200 types that vary according to risk of cervical cancer onset. HPV types 16 and 18 are the most prevalent ‘high-risk’ types, associated with 70% of cervical cancers; remaining cases are nearly all positive for other, less common high-risk HPV types. The HPV genome contains two major oncogenes, E6 and E7. The E6 protein binds and induces the degradation of p53 via a ubiquitin-mediated process. E7 protein binds and destabilizes Rb and related proteins. Together, these effects promote cell-cycle progression and viral DNA replication in differentiated keratinocytes. In pre-cancerous cervical lesions and cervical carcinomas, human papillomavirus DNA integration into the host genome may result in disruption of the viral E2 open reading frame, resulting in unregulated overexpression of HPV oncogenes E6 and E7.

Cancers that have lost Rb gene function show overexpression of p16^{INK4a}, and a reciprocal correlation between pRb and p16^{INK4a} has been established in several cancers. In most normal cells, p16^{INK4a} expression is known to be low at both the mRNA and protein levels. During HPV infection, expression of HPV E7 protein causes inactivation of Rb by binding and directing it to be degraded via a ubiquitin-proteasome pathway. Accordingly, p16^{INK4a} overexpression is observed in both established cervical cancer cell lines and human ectocervical cells immortalized by HPV types 16 and 18. In addition, upregulation of p16^{INK4a} by HPV infection has been shown to vary between high- and low-risk HPV types, with strongest upregulation by high-risk type HPV16 relative to low-risk HPV6.

The p16^{INK4a} protein has been used as an immunohistochemical and immunocytochemical marker to detect cervical cancer as follows. Some studies show very high levels of p16^{INK4a} in almost 100% of high-grade cervical dysplasias and invasive cancers, whereas no p16^{INK4a}-positive stain was found in normal cervical epithelia using the same antibodies. Further, an over-expression of p16^{INK4a} and a decreased expression of Rb have been correlated with incidence of cervical dysplasia. Recent studies indicate that mcm5 may also be a marker for the presence of cervical intraepithelial neoplasia and carcinoma but can be expressed in low grade dysplastic lesions and in some normal proliferating squamous cells. It has been demonstrated that p16^{INK4a} and mcm5 can be combined using immunological staining and flow cytometry to detect dual positive cells in a quantitative manner that tracks severity of cervical pathological state.

Cervical cancer detection at the molecular level (other protein biomarkers) has been demonstrated in several studies involving detection and quantification of cancer cell protein biomarkers by flow cytometry. Cancer biomarkers commonly detected by flow cytometry include ki67, PCNA, cyclin-D1 and cyclin-B1 and p16^{INK4a}. Most of these cancer biomarkers were primarily detected in lung, colon and breast cancer tissues. However, recent results show that increases in expression of p16^{INK4a}, mcm5 and cyclin D1 are also detected in cervical cancer samples. While this methodology is currently being developed for p16^{INK4a} mcm5 and PCNA, the detection procedure is theoretically interchangeable for any biomarker with demonstrated expression in transformed cervical epithelial cells.

As the role of human papillomavirus in cervical cancer has unfolded, it has become clear that divergence of the virus into separate genetic lineages has resulted in entities that vary in severity of host cell transformation and, ultimately, progress of disease. Five HPV genetic types are recognized as the most prevalent high-risk forms: 16, 18, 31, 33 and 45. Of these, it is widely recognized that type 16 appears in approximately half of all cervical cancers, yet HPV 18 appears to consistently contribute to poorest prognoses even though it accounts for only ˜20% of cervical cancers. An alternate way of detecting the dependence of disease severity upon HPV type shows that, when other cervical lesions are considered in addition to actual cases of cancer (ASCUS, LSIL, HSIL), it is clear that HPV types 16 and 18 vary in frequency among lesion classes in a manner reflecting the large relative risk attached to both. The combination of HPV genotyping data and pathological severity data among patients thus indicates that HPV type is information that has prognostic value for cases in which cell abnormality has begun. The problem up to this point is that an approximately 10 year latency between infection with high-risk HPV and onset of cancer prevents HPV type data by itself from being prognostically interpreted.

The Digene HPV HC2 test (Qiagen) uses a cocktail of RNA probes for 13 high risk HPV types, but a positive test result is ultimately detected by a generic antibody for DNA-RNA hybrids and does not indicate which HPV type is present. Furthermore, the HC2 test is limited by the absence of an internal control for specimen adequacy. Various PCR strategies have been used in research settings, but these usually involve nested PCR reactions that combine amplification with degenerate primers followed by secondary reactions with multiplexed, specific PCR primers.

### SUMMARY OF THE DISCLOSURE

A combination of HPV type data with detection of transformation-associated protein biomarkers is disclosed for making the HPV genotype a more prognostically relevant tool. Specifically, an assay is disclosed that combines very high sensitivity and specificity that only requires a single amplification reaction after cell sorting isolates the target cells.

In one refinement, a two-part, integrated assay is disclosed that combines flow-cytometry and multiplexed HPV genotype identification. Protein biomarker phenotype and presence of specific HPV genotype is assessed for the same cell population within a clinical sample. Its purpose is to improve the overall accuracy and specificity of detection and characterization of incipient cervical disease. The assay is compatible with samples conventionally fixed with PreservCyt (Cytyc), and can thus be applied to residual cells initially collected as part of a normal cervical lavage. High speed cell sorting recovers cells positive for over-expression of multiple protein biomarkers reported to be collectively indicative of transformation. Once recovered, these same cells are checked for high-risk HPV genotypes by using a set of PCR primers carefully designed to operate in a multiplexed reaction with no need for pre-amplification by degenerate HPV primers. Finally, detection of individual HPV types can be performed by automated detection of amplified fragment size using the capillary electrophoresis platform of the GenomeLab GeXP (Beckman Coulter).

The following oligonucleotide sequences and their reverse compliments are also disclosed, wherein the sequences by convention are written in a 5′ to 3′ bond orientation:

Sequence 1
TGGACCGGTCGATGTATGTCTTGT
Sequence 2
TACGCACAACCGAAGCGTAGAGTC
Sequence 3
AGTGTGACTCTACGCTTCGGTTGT
Sequence 4
GTGTGCCCATTAACAGGTCTTCCA
Sequence 5
ACTATAGAGGCCAGTGCCATTCGT
Sequence 6
TCGTCGGGCTGGTAAATGTTGATG
Sequence 7
CATCAACATTTACCAGCCCGACGA
Sequence 8
AAACAGCTGCTGGAATGCTCGAAG
Sequence 9
AACATAGGAGGAAGGTGGACAGGA
Sequence 10
GTGTGCTCTGTACACACAAACGAAG
Sequence 11
GAGGACACAAGCCAACGTTAAAGG
Sequence 12
GGTTCGTAGGTCACTTGCTGTACT
Sequence 13
GACAGTACCGAGGGCAGTGTAATA
Sequence 14
TACTTGTGTTTCCCTACGTCTGCGA
Sequence 15
GCGTGTGTATTATGTGCCTACGCT
Sequence 16
TTACACTTGGGTCACAGGTCGG
Sequence 17
AGGTGACACTATAGAATATGGACCGGTCGATGTATGTCTTGT
Sequence 18
GTACGACTCACTATAGGGATACGCACAACCGAAGCGTAGAGTC
Sequence 19
AGGTGACACTATAGAATAAGTGTGACTCTACGCTTCGGTTGT
Sequence 20
GTACGACTCACTATAGGGAGTGTGCCCATTAACAGGTCTTCCA
Sequence 21
AGGTGACACTATAGAATAACTATAGAGGCCAGTGCCATTCGT
Sequence 22
GTACGACTCACTATAGGGATCGTCGGGCTGGTAAATGTTGATG
Sequence 23
AGGTGACACTATAGAATACATCAACATTTACCAGCCCGACGA
Sequence 24
GTACGACTCACTATAGGGAAAACAGCTGCTGGAATGCTCGAAG
Sequence 25
AGGTGACACTATAGAATAAACATAGGAGGAAGGTGGACAGGA
Sequence 26
GTACGACTCACTATAGGGAGTGTGCTCTGTACACACAAACGAAG
Sequence 27
AGGTGACACTATAGAATAGAGGACACAAGCCAACGTTAAAGG
Sequence 28
GTACGACTCACTATAGGGAGGTTCGTAGGTCACTTGCTGTACT
Sequence 29
AGGTGACACTATAGAATAGACAGTACCGAGGGCAGTGTAATA
Sequence 30
GTACGACTCACTATAGGGATACTTGTGTTTCCCTACGTCTGCGA
Sequence 31
AGGTGACACTATAGAATAGCGTGTGTATTATGTGCCTACGCT
Sequence 32
GTACGACTCACTATAGGGATTACACTTGGGTCACAGGTCGG

In another refinement, a sequential method is disclosed wherein cells exhibiting over-expression of one or more protein biomarkers are captured, via cell sorting of conventionally fixed and immunologically stained cervical cell populations, and then directly checked for human papillomavirus content. The method is categorically hierarchical and allows specific HPV genotype(s) to be attributed to a cell subpopulation that has already been characterized for protein biomarker phenotype. In this way cells are detected that individually exhibit combined risk factors for cervical disease, specifically indicating the presence of cell lineages that contain both protein biomarker indication of disease state and presence of specific, high-risk HPV type(s). Without being bound to any particular theory, the cell sorting specifically acts as: 1) a method for rare event detection that proportionally quantifies occurrence of biomarker over-expression within a cervical cell population, and 2) a pre-filtering workflow that channels only abnormal cells toward the HPV assay. Within the disclosed method, gating criteria for cell sorting via flow cytometry may be set to isolate cells exhibiting over-expression of any two proteins among the set including, but not limited to, p16^{INK4a}, mcm5, PCNA, and any other protein biomarker associated with cervical epithelial cell transformation.

In a refinement, any or all of Sequences 17-32 are used to perform specific detection and identification of individual high-risk HPV types 16, 18, 31, 33, 45 and/or 52 by executing a single multiplexed PCR amplification directly from cells captured during cell sorting. Further, in this claim, individual HPV types are subsequently identified by resolving target amplicons via automated capillary electrophoresis.

In a refinement, any or all of Sequences 1-16, end-labeled with a fluorescent dye, are used to perform specific detection and identification of individual high-risk HPV types 16, 18, 31, 33, 45 and/or 52 by executing a single multiplexed PCR amplification from cells captured during cell sorting. In this claim, individual HPV types can subsequently be identified by resolving target amplicons via automated capillary electrophoresis. Alternately, in this claim, individual HPV types are represented by target amplicons of sufficiently different size so that detection can be performed with standard agarose gel electrophoresis.

In a refinement, sensitivity and specificity of HPV detection and identification is enhanced by using a thermocycling program optimized according to criteria normally used for qPCR, in which there are only two cycle steps that respectively perform denaturation and annealing/extension during extremely short incubation times for ≧35 cycles.

A method is disclosed for constructing a clinical prognosis protocol based upon a matrix of protein biomarker phenotypes versus HPV types as determined via the methods described above. Protein biomarker phenotypes in this case specifically include both the particular protein biomarkers detected and their proportions within the cell population as determined by positive versus negative sorting events automatically counted during the cell sorting process. The combined protein and HPV type characterizations of individual clinical samples, obtained via the assays described above, are clustered with pathology data from case histories to produce a finely resolved association matrix for assay results versus predicted disease outcome.

Other advantages and features will be apparent from the following detailed description when read in conjunction with the attached drawings.

### BRIEF DESCRIPTION OF THE DRAWINGS

For a more complete understanding of the disclosed methods and apparatuses, reference should be made to the embodiments illustrated in greater detail in the accompanying drawings, wherein:

FIG. 1 is a flow diagram for the cell sorting stage of a disclosed assay;

FIG. 2 is a flow diagram for the PCR and capillary electrophoresis stage of a disclosed assay;

FIG. 3 illustrates, graphically, clinically normal cervical cells characterized by signal intensity for immunofluorescent staining with antibodies for the human proteins p16^{INK4a} and MCM5;

FIG. 4 illustrates, graphically, low-grade squamous intraepithelial lesion cells characterized by signal intensity for immunofluorescent staining with antibodies for the human proteins p16^{INK4a} and MCM5;

FIG. 5 illustrates, graphically, high-grade squamous intraepithelial lesion cells characterized by signal intensity for immunofluorescent staining with antibodies for the human proteins p16^{INK4a} and MCM5;

FIG. 6 illustrates, illustrates, graphically, HeLa cells characterized by signal intensity for immunofluorescent staining with antibodies for the human proteins p16^{INK4a} and MCM5;

FIG. 7 illustrates, graphically, percent signal type by sample class across samples illustrated in FIGS. 3-6;

FIG. 8 shows test results that demonstrate high sensitivity and specificity for the disclosed detection of HPV from pre-sorted cells; and

FIG. 9 shows test results that demonstrate proof of concept for detection of fluorescence-tagged HPV amplicons, from multiplex PCR with fluorescence-tagged primers, using an automated capillary electrophoresis platform.

It should be understood that the drawings are not necessarily to scale and that the disclosed embodiments are sometimes illustrated diagrammatically and in partial views. In certain instances, details which are not necessary for an understanding of the disclosed methods and apparatuses or which render other details difficult to perceive may have been omitted. It should be understood, of course, that this disclosure is not limited to the particular embodiments illustrated herein.

### DETAILED DESCRIPTION OF THE PRESENTLY PREFERRED EMBODIMENTS

A two part assay is disclosed that enables collection of both protein biomarker phenotype and specific HPV genotype data from within a clinically derived population of cervical epithelial cells. Data is collected hierarchically. Presence of multiple transformation-associated protein biomarkers acts as a gating criterion for cell sorting, followed by application of a PCR protocol sensitive enough to detect and identify individual HPV types from within the cells captured during sorting. The workflow has been optimized to work with cells conventionally fixed in PreservCyt (Cytyc), and it can be performed on residual cells remaining in a stored sample after a Pap test has been performed.

Protein biomarker data is quantified proportionally within a cell population by counting positive versus negative sorting events. Thus biomarker data for a clinical sample is not a single value representing an overall staining intensity, but is instead a value reflecting the proportion of the cell population in which individual cells surpass an intensity threshold for each of two or more biomarkers. The gating criterion for a positive sorting event can be set as a combination of desired signal intensities for the protein biomarkers being used. Biomarkers are detected through conventional immunological staining of fixed cells with fluorescently labeled antibodies, and can include, but are not limited to, proteins of reported association with transformation of cervical epithelial cells, such as p16^{INK4a} and mcm5, and other proteins associated with progression of other malignancies, such as PCNA, caPCNA, mcm2, etc. Cells that pass the gating criterion are sorted directly into 0.2 ml PCR wells (either as individual tubes or within 96 well PCR plates).

HPV detection and identification is performed by using a multiplexed PCR, using sorted cells directly as template with no further sample preparation. This reaction uses primer pairs that have been carefully designed according to three general criteria.

Primer pairs were designed to avoid covering annealing sites with positions known to be polymorphic among isolates of an HPV type. For each HPV type, all available isolates reported to GenBank, that included sequence for genes E6 and/or E7, were used to produce separate sequence alignments for each of those two genes. Alignments were assembled using sequence alignment tools such as ClustalX in order to align the open reading frames after virtual translation to amino acid sequence. Gaps were inserted manually for maximum preservation of homologous alignment of amino acid positions, after which gaps were then inserted into DNA versions of aligned sequences. Sequence positions that exhibited polymorphism within an HPV type were noted and tagged as “excluded regions” when the contiguous E6-E7 query sequence was submitted to primer design tools such as PrimerQuest^{SM} (Integrated DNA Technologies) for primer design. In cases where potential primer sites with optimal kinetic properties were limited in number, primers were allowed to cover one polymorphic site only in the 5′ half of the primer sequence. In this way, 3′ end stability should be preserved and the effect on overall sensitivity should be negligible. Tables 4A and 4B list the GenBank accession numbers for all HPV sequences used to produce within-type sequence alignments for genes E6 and E7. Finally, for a single HPV type, the query sequence submitted to PrimerQuest^{SM} consisted of a contiguous fragment covering the viral genome from the 5′ end of E6 to the 3′ end of E7. For each HPV type, this fragment was taken from the reference whole genome sequence submitted for that type. For HPV52, E7 was excluded from the design query because no isolates were available for detecting polymorphism. GenBank accession numbers for the whole genome reference sequences used for each type appear as the top accession number listed in each column in Tables 4A and 4B. PrimerQuest^{SM} was instructed to return 50 potential primer pairs for each query so that a final multiplex could be assembled by picking primer pairs that maximized sequence divergence among HPV types at annealing sites.

All primers were designed to be as similar as possible with regard to kinetic properties. Tₘ values were constrained within a range covering four degrees Celsius, with a difference no greater than two degrees allowed between any two primers of a matched pair. Primer length ranges from 22 to 25 nucleotides, with 13 of 16 all being 24 nucleotides in length. (Sequences 17-32 are just Sequences 1-16 with an 18-mer and 19-mer universal priming sequence attached—see description of the preferred detection embodiment of the invention below.) GC % ranges from 48% to 54%. 3′ end stability scores range from approximately 3 to 7, according to the algorithm used by PrimerQuest^{SM}. If Sequences 1-16 are used, all target amplicons are less than 300 bp. For Sequences 17-32, all target amplicons are less than 350 bp. When combined with a high performance recombinant DNA polymerase such as Platinum PFX (Invitrogen), allows for the use of stringent thermocycling parameters that mimic programs used for qPCR. This second design aspect simultaneously increases sensitivity and specificity, and contributes to the successful use of a single PCR to detect and identify individual HPV types in a multiplexed context.

Finally, a multiple sequence alignment of the contiguous E6 and E7 open reading frames was assembled for HPV types 16, 18, 31, 33, 45 and 52. The sequences were again taken from the reference whole genome sequences submitted to Genbank for each HPV type. Alignment was done by first translating the reading frames in GeneDoc 2.6.002 and then assembling alignments of the amino acid sequences in ClustalX. In this way all gaps are introduced in frame, and evolutionarily homologous positions are more likely to be aligned. Nucleotide sequences were then aligned manually to match the amino acid alignments by using the gap insertion and sliding functions in GeneDoc. Primer pairs for each HPV type were chosen by comparing annealing sites with homologous regions among all six aligned sequences. Primer pairs that covered annealing sites most divergent from other HPV types were chosen with specific attention to sequence divergence near the 3′ end of each primer. Simultaneously, primer pairs were chosen so that the target amplicons for all HPV types differ from each other by at least 7 bp. Table 2 lists Sequences 1-16 with their actual primer names and salient characteristics. Table 3 simply lists Sequences 17-32 with their primer names and target amplicon lengths.

Final detection of amplicons from the multiplexed PCR is performed by using an automated parallel capillary electrophoresis platform such as the GenomeLab GeXP Genetic Analysis System (Beckman Coulter). The PCR master mix contains two fluorescent dye-labeled oligonucleotides that act as universal primers. The sequences of these oligonucleotides are complementary to, respectively, the 18 nucleotide sequence common to the 5′ end of Sequences 17, 19, 21, 23, 25, 27, 29 and 31, and the 19 nucleotide sequence common to the 5′ end of Sequences 18, 20, 22, 24, 26, 28, 30 and 32. Use of Sequences 17-32 in conjunction with the PCR master mix of the GenomeLab GeXP Start Kit allows for a kinetic turnover in which the HPV type-specific primers dominate target amplification for approximately the first three cycles, followed by a shift to dominance of priming by the dye-labeled universal primers. Detection and analysis is then performed using standard fragment analysis with the GenomeLab DNA Size Standard Kit-400.

Sequences 1-16, directly end-labeled with a fluorescent dye, may be used to perform the multiplex PCR followed by amplicon detection via an automated parallel capillary electrophoresis platform such as the GenomeLab GeXP Genetic Analysis System (Beckman Coulter). This alternate method does not require use of the universal primers included in the PCR master mix of the GenomeLab GeXP Start Kit. Amplicons may alternately be detected by standard fragment size analysis via agarose gel electrophoresis.

Gel resolution is enhanced by incorporating 50% Synergel (Diversified Biotech) with conventional molecular biology grade agarose. Assay performance is optimized for execution with Platinum PFX DNA polymerase (Invitrogen), following the thermocycling protocol listed below:

Step 195° C. for 2 minutes initial denaturation/cell lysis

Step 2 95° C. for 15 seconds

Step 3 62° C. for 5 seconds

Step 4 72° C. for 5 seconds

Step 5 GOTO Step 2 39X

Step 6 4° C. hold

TABLE 2
HPV targeted primers without universal priming sequence

| | | Start | Tm | ES | PP | PS |
|---|---|---|---|---|---|---|
| HPV16E6E7F | TGGACCGGTCGATGTATGTCTTGT | 415 | 60.0° C. | 5.17 | 5.12 | 272 bp |
| HPV16E6E7R | TACGCACAACCGAAGCGTAGAGTC | 686 | 60.9° C. | 5.32 | | |
| HPV18E6E7F | ACTATAGAGGCCAGTGCCATTCGT | 398 | 60.0° C. | 5.91 | 0.42 | 247 bp |
| HPV18E6E7R | TCGTCGGGCTGGTAAATGTTGATG | 644 | 59.6° C. | 5.08 | | |
| HPV31E6E7F | AACATAGGAGGAAGGTGGACAGGA | 379 | 59.4° C. | 5.87 | 5.2 | 291 bp |
| HPV31E6E7R | GTGTGCTCTGTACACACAAACGAAG | 669 | 58.4° C. | 5.75 | | |
| HPV33E6E7F | GAGGACACAAGCCAACGTTAAAGG | 469 | 58.5° C. | 5.12 | 2.89 | 230 bp |
| HPV33E6E7R | GGTTCGTAGGTCACTTGCTGTACT | 698 | 58.6° C. | 4.74 | | |
| HPV45E6E7F | GACAGTACCGAGGGCAGTGTAATA | 395 | 58.1° C. | 3.04 | 4.97 | 80 bp |
| HPV45E6E7R | TACTTGTGTTTCCCTACGTCTGCGA | 474 | 59.9° C. | 7.16 | | |
| HPV52E6E7F | GCGTGTGTATTATGTGCCTACGCT | 182 | 59.5° C. | 7.13 | 7.82 | 266 bp |
| HPV52E6E7R | TTACACTTGGGTCACAGGTCGG | 447 | 59.2° C. | 6.75 | | |

Start=5′ starting position within the reference sequence used as the target for primer design for each HPV type. Reverse primers are the reverse compliment of the annealing site within the reference sequence, so their starting position occurs at the 3′ end of the annealing site relative to the reference sequence. ES=end stability. PP=pair penalty. PS=product size. ES and PP are as reported by PrimerQuest^{SM} (Integrated DNA Technologies).

TABLE 3
HPV targeted primers with added universal priming sequence

| | | Product Size |
|---|---|---|
| HPV16E6E7FU | AGGTGACACTATAGAATATGGACCGGTCGATGTATGTCTTGT | 309 |
| HPV16E6E7RU | GTACGACTCACTATAGGGATACGCACAACCGAAGCGTAGAGTC | |
| HPV18E6E7FU | AGGTGACACTATAGAATAACTATAGAGGCCAGTGCCATTCGT | 284 |
| HPV18E6E7RU | GTACGACTCACTATAGGGATCGTCGGGCTGGTAAATGTTGATG | |
| HPV31E6E7FU | AGGTGACACTATAGAATAAACATAGGAGGAAGGTGGACAGGA | 328 |
| HPV31E6E7RU | GTACGACTCACTATAGGGAGTGTGCTCTGTACACACAAACGAAG | |
| HPV33E6E7FU | AGGTGACACTATAGAATAGAGGACACAAGCCAACGTTAAAGG | 267 |
| HPV33E6E7RU | GTACGACTCACTATAGGGAGGTTCGTAGGTCACTTGCTGTACT | |
| HPV45E6E7FU | AGGTGACACTATAGAATAGACAGTACCGAGGGCAGTGTAATA | 117 |
| HPV45E6E7RU | GTACGACTCACTATAGGGATACTTGTGTTTCCCTACGTCTGCGA | |
| HPV52E6E7FU | AGGTGACACTATAGAATAGCGTGTGTATTATGTGCCTACGCT | 303 |
| HPV52E6E7RU | GTACGACTCACTATAGGGATTACACTTGGGTCACAGGTCGG | |

Start=5′ starting position within the reference sequence used as the target for primer design for each HPV type, as in Table 2. The additional universal priming sequence specifically allows the assay to incorporate automated detection of target amplicons by using a Genome Lab GeXP (Beckman Coulter).

TABLE 4A
Genbank accession numbers of isolates used for SNP exclusion
within HPV types 16, 18 and 31.

| HPV16 | | HPV18 | | HPV31 | |
|---|---|---|---|---|---|
| E6 | E7 | E6 | E7 | E6 | E7 |
| NC_001526 | NC_001526 | NC_001357 | NC_001357 | J04353 | J04353 |
| EF122273 | EU118173 | EF422111 | EF422144 | FJ202003 | EF422156 |
| EF122274 | AY686584 | EF422110 | EF422143 | FJ202002 | EF422155 |
| EF122275 | AY686583 | EF422109 | EF422142 | EF422123 | EF422154 |
| EF122276 | AY686582 | EF202155 | EF202143 | EF422122 | EF422153 |
| EF122277 | AY686581 | EF202154 | EF202144 | EF422121 | EF422152 |
| EF122278 | AY686580 | EF202153 | EF202145 | EF422120 | EF422151 |
| EF122279 | AY686579 | EF202152 | EF202146 | EF422119 | EF422150 |
| EF122280 | AF536180 | EF202143 | EF202147 | EF566933 | EF422149 |
| EF122281 | AF536179 | EF202144 | EF202148 | | EF422148 |
| EF122282 | AF534061 | EF202145 | EF202149 | | EF422147 |
| EF122283 | AF402678 | EF202146 | EF202150 | | EF422146 |
| EF122284 | AF472509 | EF202147 | EF202151 | | EF422145 |
| EF122285 | AF472508 | EF661657 | EF202152 | | |
| EF122286 | AF125673 | EF661656 | EF202155 | | |
| EF122287 | EF422141 | EF202148 | EF202154 | | |
| | EF422140 | EF202149 | EF202153 | | |
| | EF422139 | EF202150 | AY262282 | | |
| | EF422138 | EF202150 | X05015 | | |
| | EF422137 | EF661655 | U89349 | | |
| | EF422136 | EF661654 | Y18493 | | |
| | EF422135 | | Y18492 | | |
| | EF422134 | | Y18491 | | |
| | EF422133 | | A06324 | | |
| | EF422132 | | PPH18A | | |
| | EF422131 | | | | |
| | EF422130 | | | | |
| | EF422129 | | | | |
| | EF422128 | | | | |

In each case shown in Table 4A, the accession number listed at the top is the complete genome sequence for its respective HPV type. The query sequence for primer design in each case is the contiguous E6-E7 sequence copied from within the complete genome sequence. Separate alignments for genes E6 and E7 within a type were assembled using ClustalX. All variable sites observed among isolates within a type were subsequently excluded from primer designs where feasible. Where variable sites could not be avoided, primers were chosen that covered no more than one variable site near their 5′ end.

TABLE 4B
Genbank accession numbers of isolates used for SNP exclusion
within HPV types 33, 45 and 52

| HPV33 | | HPV45 | | HPV52 |
|---|---|---|---|---|
| E6 | E7 | E6 | E7 | E6 |
| M12732 | M12732 | X74479 | X74479 | X74481 |
| EF422127 | A12360 | EF202167 | M38198 | FJ002423 |
| EF422126 | A07020 | EF202166 | Y13218 | EF566936 |
| EF422125 | EF422157 | EF202165 | EF202167 | EF566935 |
| EF422124 | EF422158 | EF202160 | EF202166 | EF566934 |
| M12732 | | EF202159 | EF202165 | EF566923 |
| A12360 | | EF202158 | EF202164 | EF566922 |
| A07020 | | EF202157 | EF202163 | DQ057295 |
| EF566921 | | EF202156 | EF202162 | DQ057294 |
| EF566920 | | Y13218 | EF202161 | DQ057293 |
| | | M38198 | EF202160 | DQ057292 |
| | | | EF202159 | DQ057291 |
| | | | EF202158 | DQ057290 |
| | | | EF202157 | |
| | | | EF202156 | |
| | | | AJ242956 | |

In each case shown in Table 4B, the accession number listed at the top is the complete genome sequence for its respective HPV type. Use of E6 and E7 sequences and performance of alignments is as in Table 4A. In the case of HPV52, only gene E6 is represented by multiple isolates, so primer design was restricted to E6.

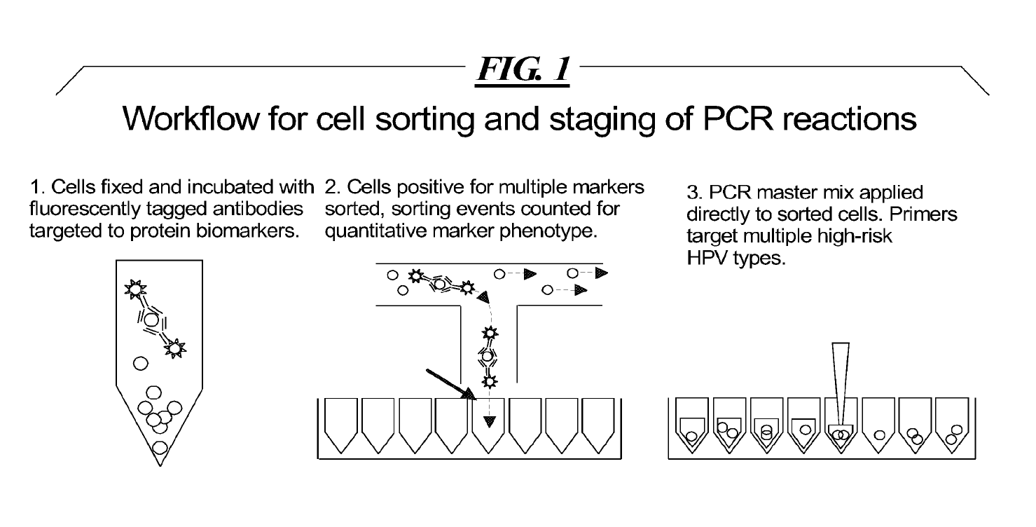

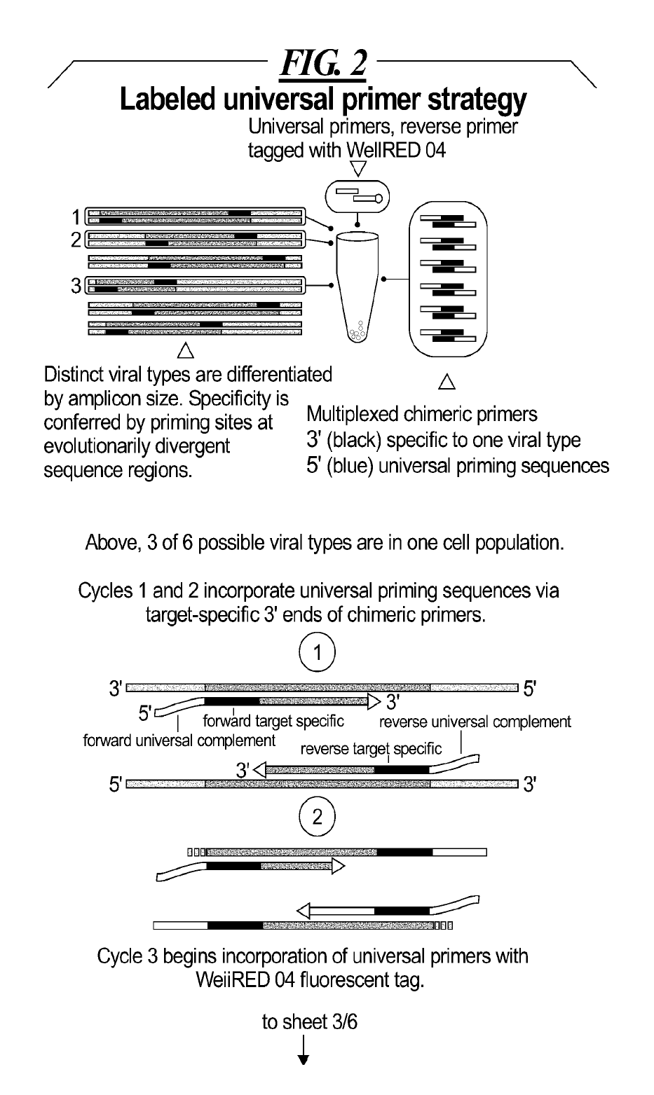

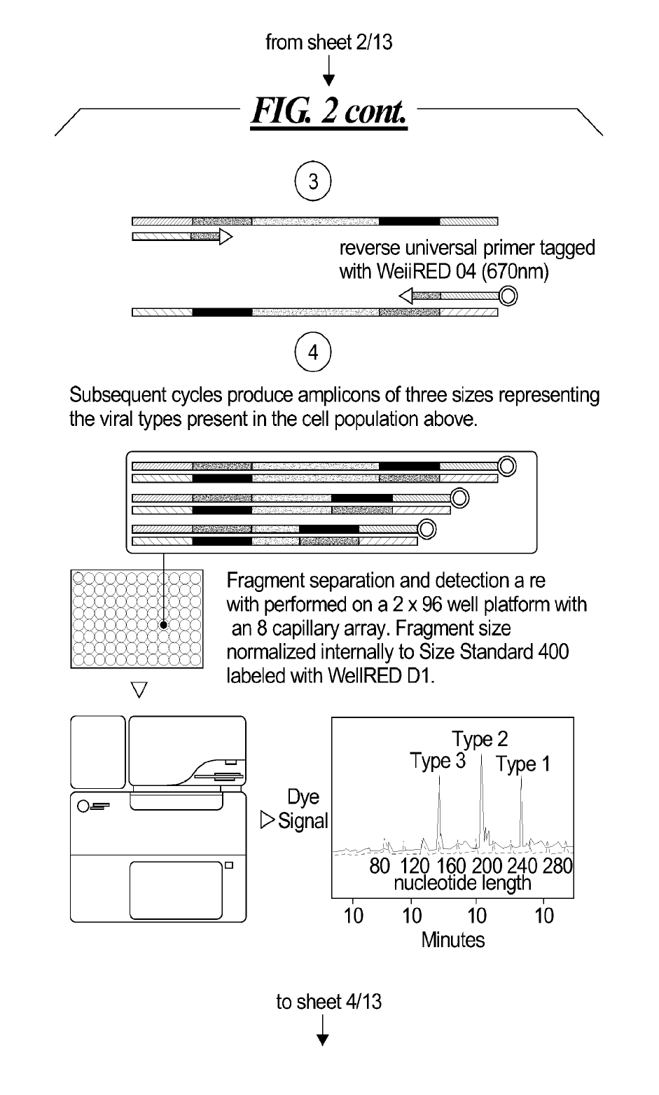

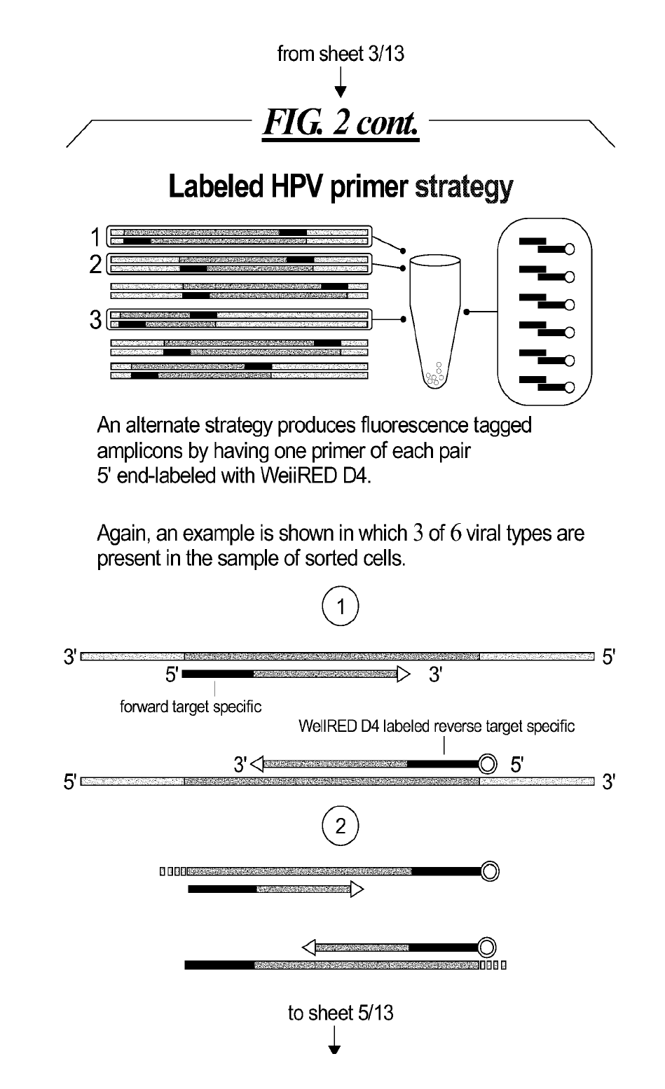

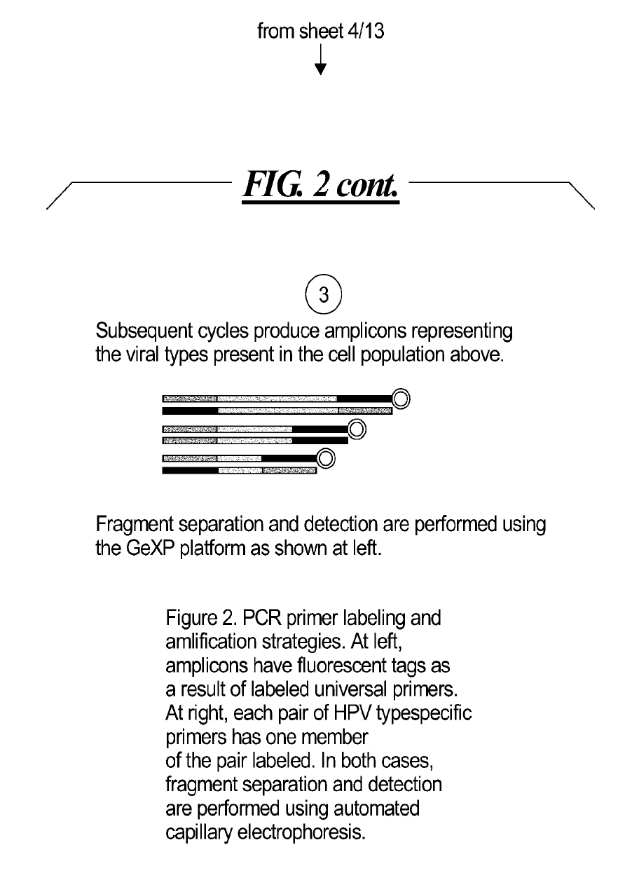

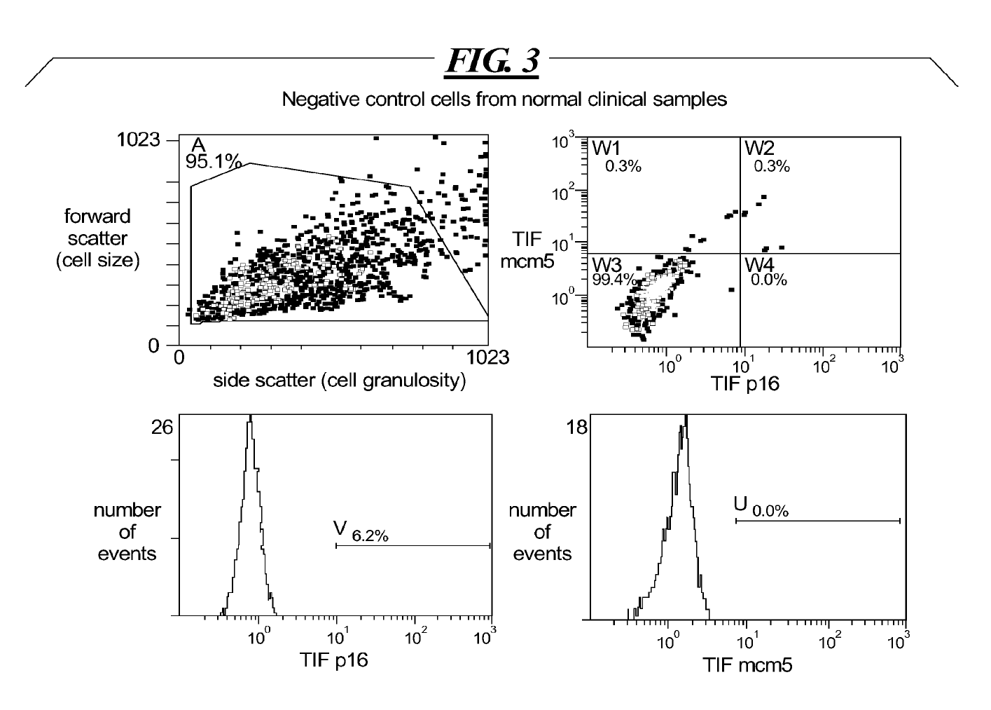

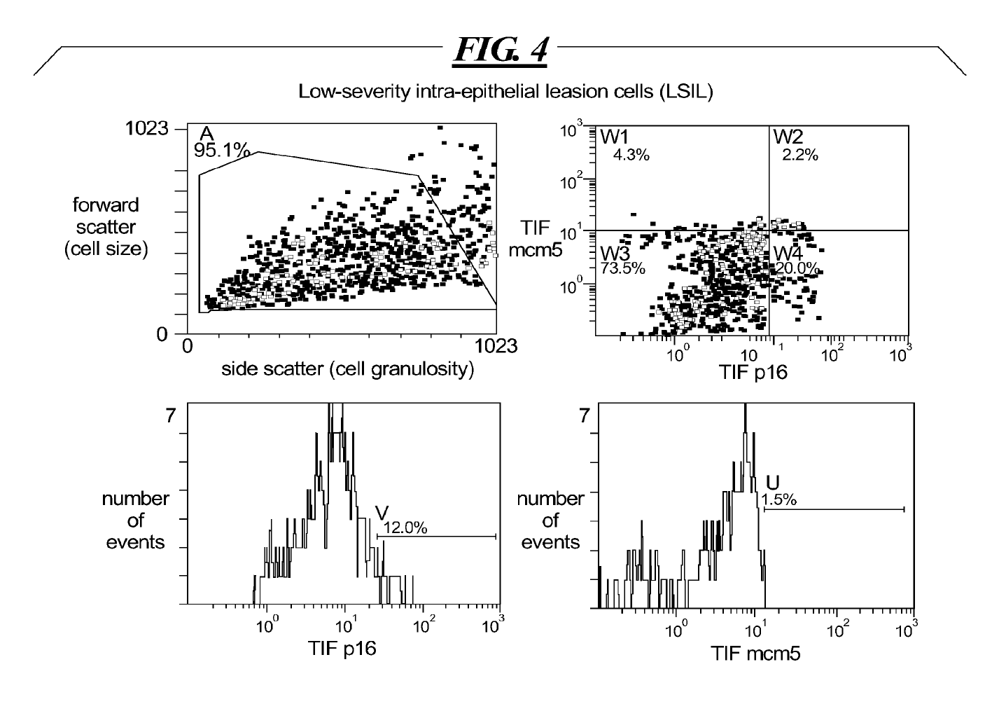

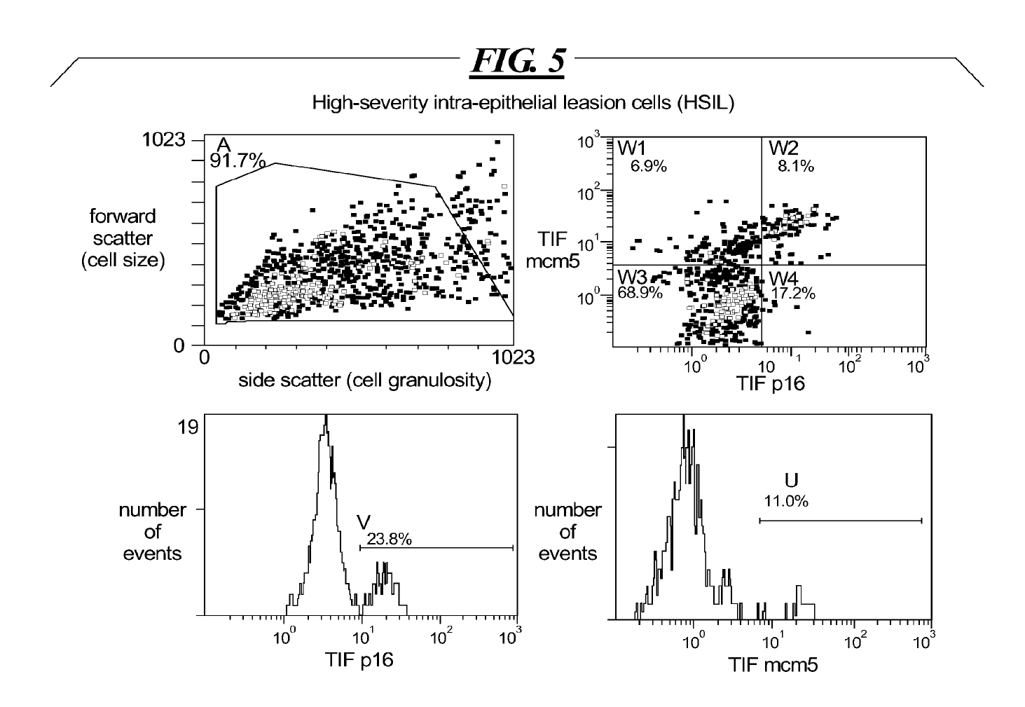

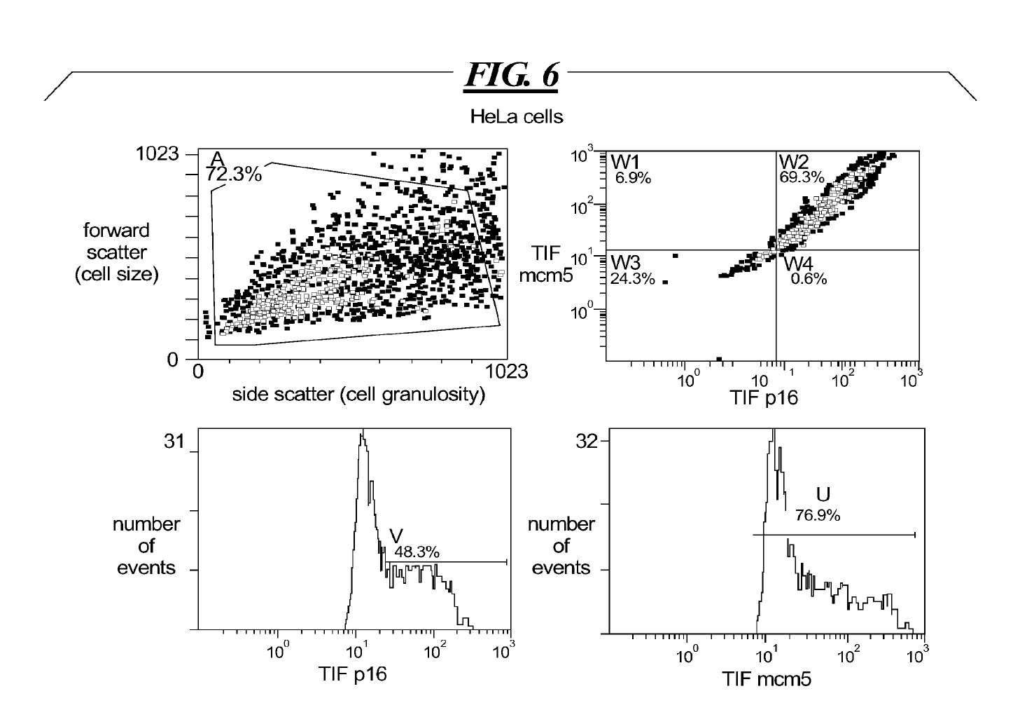

Flow cytometry detection of dysplastic cells from clinical samples using immuno-staining for p16 and mcm5 is disclosed. Flow cytometry can detect dual positivity for overexpression of p16 and mcm5 in a manner that tracks cytomorphological classification of clinical samples. Cell populations from clinical samples classified as normal, LSIL and HSIL were analyzed, as well as HeLa cells. FIGS. 1-5 illustrate flow cytometry results for all cell populations. All cells were fixed in PreservCyt (Cytyc, 90% MeOH/10% H20). Cells were incubated with p16/FITC and mcm5/APC antibodies. Cell samples were gated on forward/side scatter diagram (upper left panel of FIGS. 2-5). Gated cells were analyzed for levels of p16 and mcm5. The dual parameter diagram (upper right panel of FIGS. 2-5) shows percentages of cells negative or positive for either marker. Percentages of cells positive for each marker independently are shown in the lower left and right panel of FIGS. 2-5. Three independent experiments were performed for each tissue type. FIG. 6 summarizes the percentage of cells displaying each signal type within each tissue type.

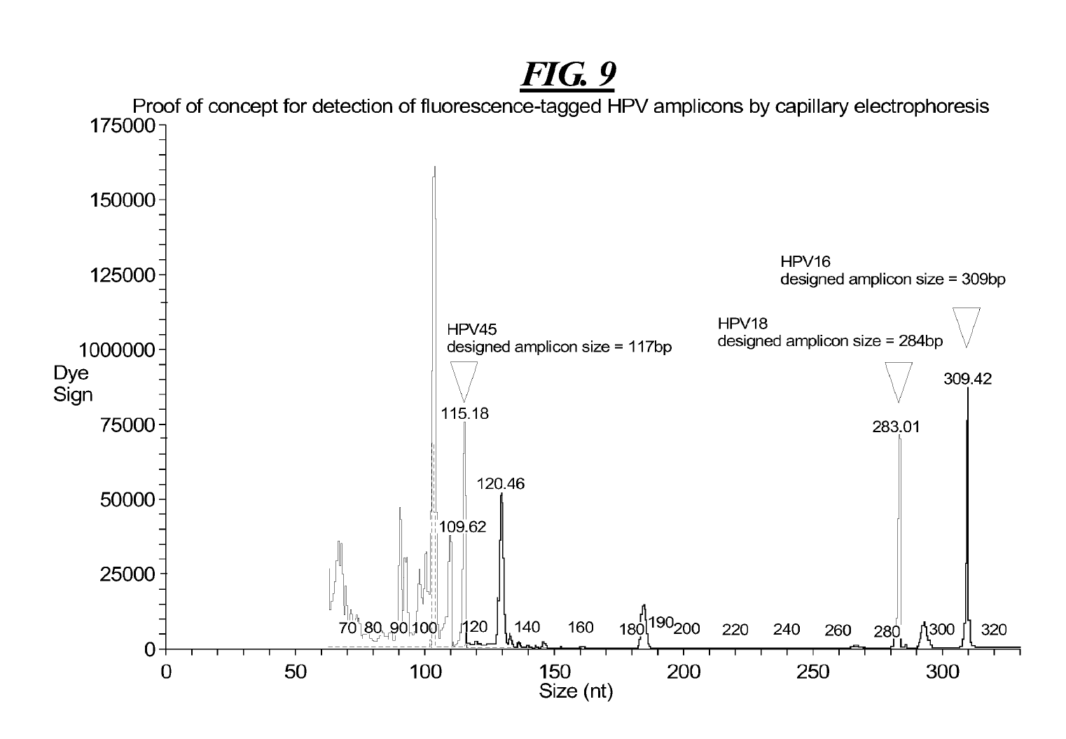

In FIG. 8, multiplexed PCR detection of HPV16, HPV18 and HPV45 with high sensitivity and specificity is demonstrated. Proof of principle is demonstrated with a 3-plex of PCR primer pairs. Primers were designed to produce amplicons of 272 bp, 247 bp and 80 bp respectively for HPV 16, HPV 18 and HPV45. All amplicons fall within the contiguous region of the E6 and E7 open reading frames. The design strategy explicitly focused on achieving robust detection within each type while avoiding non-specific amplification. Within each HPV type, multiple sequence alignments of E6 and E7 were performed for isolates culled from the NCBI Nucleotide database in order to detect polymorphisms among clinical isolates. Tables 4A and 4B include the Genbank accession numbers for all isolates used to perform alignments for HPV 16, HPV 18 and HPV45. Primers were designed using PrimerQuest^{SM} (IDT). This design application allows exclusion of positions within the target sequence, ensuring that polymorphic sites do not affect annealing. Primer pairs were chosen so that GC %, Tₘ and 3′ end stability values were as similar as possible, resulting in similar sensitivity of detection for separate HPV types within a multiplexed reaction. Finally, the primer sets were chosen to cover locations within E6-E7 that are highly divergent between any target HPV type and the others. Table 5 lists Tₘ and amplicon size for the primer pairs used in this 3-plex.

TABLE 5
Properties of PCR primer
pairs targeted to HPV18, HPV16 and
HPV45

| | Tₘ | Target Size |
|---|---|---|
| HPV18E6E7F | 60.0° C. | 247 bp |
| HPV18E6E7R | 59.6° C. | |
| HPV16E6E7F | 60.0° C. | 272 bp |
| HPV16E6E7R | 60.9° C. | |
| HPV45E6E7F | 58.1° C. | 80 bp |
| HPV45E6E7R | 59.9° C. | |

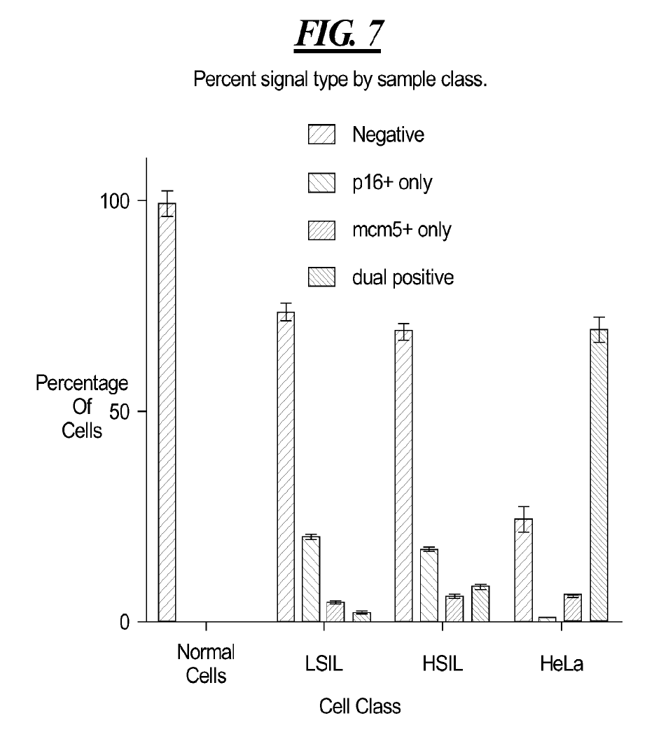

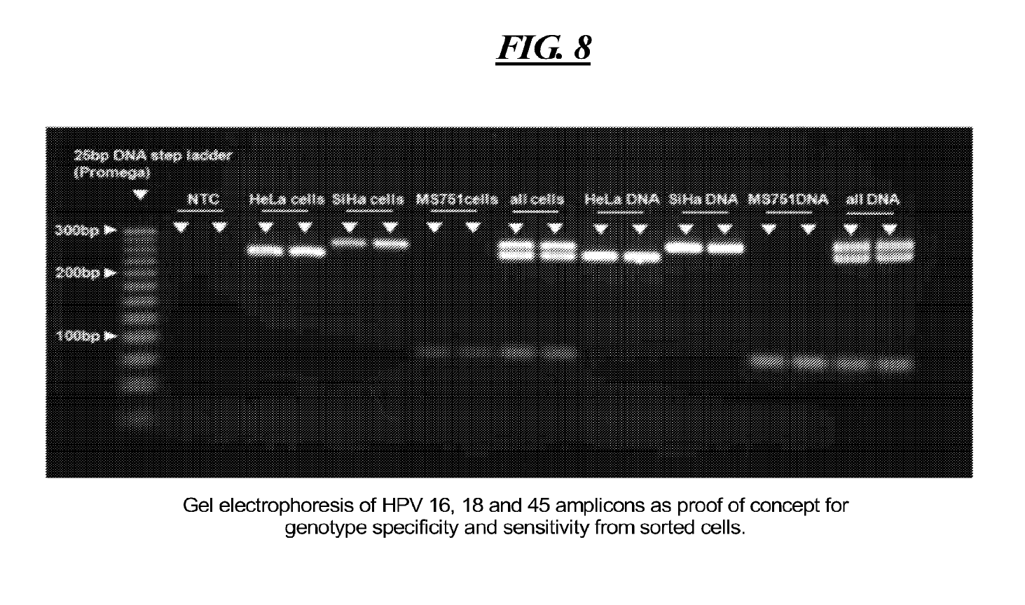

The specificity and sensitivity of the HPV16/18/45 3-plex were tested using cell lines HeLa, SiHa and MS751, which respectively contain integrated genomic copies of HPV18, HPV16 and HPV45. Cells were first fixed in 90% methanol, as would normally happen if a clinical sample were fixed in PreservCyt (Cytyc). Cells were incubated with p16/FITC and mcm5/APC antibodies and sorted as above. Bulk sorted cells were resuspended in 90% methanol. Suspensions were made with 20 cells per microliter of a single type and 20 of each cell type per microliter. Sorted cells were used directly as PCR template by placing 1 μl aliquots of cell suspensions in 0.2 ml PCR tubes and drying in a Speed Vac (Savant) with no heat. 25 μl aliquots of PCR master mix were placed directly on top of dried cells. In parallel, as positive controls to rule out amplification artifacts from cell material, an identical set of amplifications was performed using genomic DNA purified from the same cell lines. Positive control reactions contained 10 ng of one DNA type (lanes 12-17) or 10 ng of each DNA type (lanes 18-19). FIG. 7 demonstrates high specificity and specificity of HPV detection. First, when directed against ˜20 cells of each cell line individually (lanes 4-9), the multiplex of three primer pairs only produces an amplicon of the correct target size for the HPV type expected in each case. Second, in all of those reactions, no undesigned amplicons appear as a result of nonspecific amplification from human genomic material. Third, when all three cell lines are mixed, the 3-plex is able to detect all three HPV types with the same visible intensity, showing that amplification efficiencies of targets are not interfered with by the presence of multiple HPV types in a reaction. Reactions using purified cell line genomic DNA produce an identical set of amplicons and relative intensities, so other cell-derived materials are not producing amplification artifacts. Finally, these tests used a thermocycling program that ran for 40 cycles in order to see whether a large number of cycles would be permitted to increase sensitivity. The fact that these clear results occur even after 40 cycles indicates the robust specificity of the primer design.

In FIG. 7: Lane 1, 25 bp DNA stepladder (Promega); Lanes 2-3, no template control—25 μl aliquots of PCR master mix with 1 μl H₂O as mock template addition; Lanes 4-9 replicate pairs of the 3-plex primer set applied to each cell line individually (each reaction contains ˜20 cells that have passed through the immunostaining and sorting process); Lanes 10-11—two replicates of the 3-plex primer set applied to all three cell lines mixed together (˜20 cells of each line); Lanes 12-19—purified genomic DNA from the three cell lines used to reproduce the same pattern of template types as lanes 4-11; Single cell type reactions (lanes 12-17) contained 10 ng genomic DNA from each respective cell line, while lanes 18 and 19 show two replicates of amplification from a template mix with 10 ng of each DNA type. Gel is 1.25% agarose, 1.25% Synergel (Diversified Biotech) in 1X TAE. Separation performed at 70V for approximately 30 minutes. Gel post-stained with ethidium bromide.

Proof of principle for detection of amplicons via the capillary electrophoresis platform is demonstrated by using chimeric versions of the same primer 3-plex to which the universal primer complementary sequences have been added. These chimeric primers correspond to Sequences 17, 18, 21, 22, 29 and 30 in claim 1. These primers were targeted at template consisting of a mix of purified DNA from HeLa, SiHa and MS751 cells, 10 ng of each per 1 μl of solution. Amplification was performed using the 5×PCR buffer included in the GenomeLab GeXP Start Kit (Beckman Coulter, Inc.), which includes forward and reverse universal primers complementary to the 5′ ends of the chimeric primers. The reverse universal primer is 5′ end-labeled with We11RED D4, a far-red fluorescent dye with an emission peak at 670 nm. Three replicate reactions were performed for both the positive detection trial and a no template control condition in which water was added in place of template DNA. A three-phase thermal cycling program was used to exploit the three different annealing conditions that occur sequentially when using this PCR chemistry (see the left side of FIG. 2 for an illustration of the three annealing events). The thermal cycling program is as follows:

1 95° C. 10 minutes
2 94° C. 15 seconds
3 65° C. 20 seconds
4 72° C. 30 seconds

### 5 GOTO 2 2×

6 94° C. 15 seconds
7 72° C. 1 minute

### 8 GOTO 6 4×

9 94° C. 30 seconds
10 55° C. 30 seconds
11 70° C. 1 minute

### 12 GOTO 9 34×

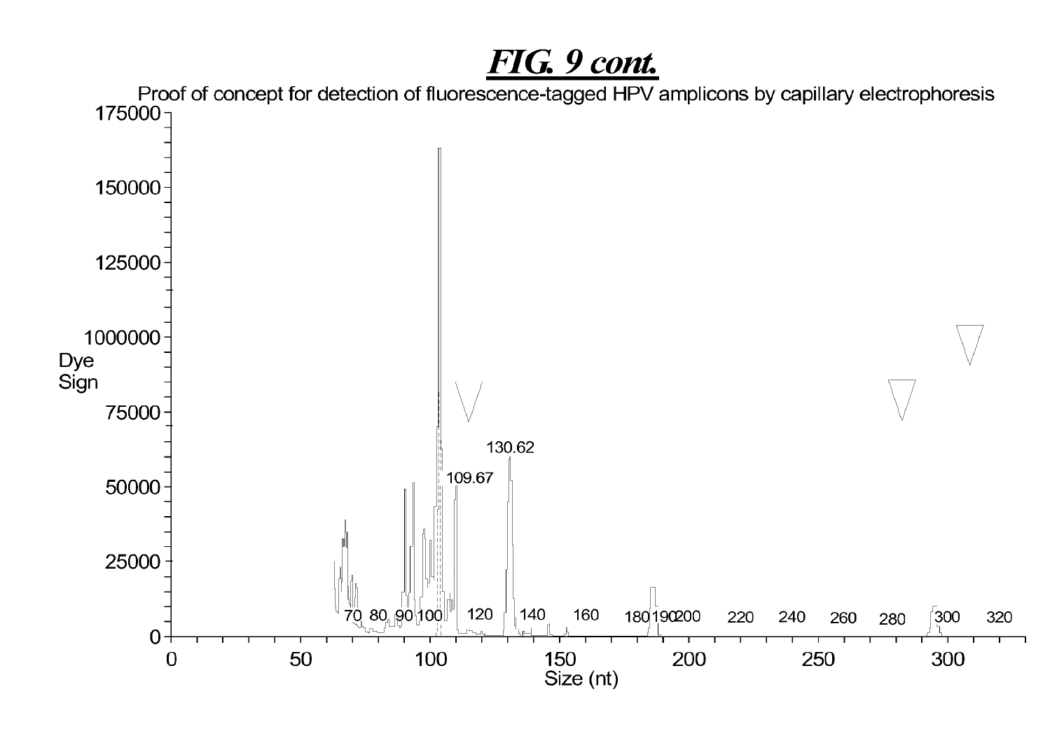

13 4° C. hold
Following thermal cycling, samples for fragment analysis were produced by combining 0.5 μl PCR reaction with 39 μl GeXP Sample Loading Solution (Beckman Coulter, Inc.) and 0.5 μl DNA Size Standard—400 (Beckman Coulter, Inc.). Fragment separation samples were prepared for each replicate of both the positive detection trial and the no template control. Fragment separation and detection were performed using the method “Frag-3” within the device controller software for the GenomeLab GeXP Genetic Analysis System. FIG. 9 illustrates successful detection of electropherogram peaks specific to predicted amplicon sizes for HPV16, 18 and 45. All three peaks are absent from the no-template control.

While only certain embodiments have been set forth, alternatives and modifications will be apparent from the above description to those skilled in the art. These and other alternatives are considered equivalents and within the spirit and scope of this disclosure.

1. A method for detecting high-risk HPV genotypes in a cervical cell sample, the method comprising:
collecting the sample from a cervical lavage or a Pap test;
fixing cells of the sample with fluorescently tagged antibodies targeted to multiple biomarkers;
separating cells positive for multiple biomarkers from cells positive for one or less biomarkers;
applying a polymerase chain reaction (PCR) master mix to the cells positive for multiple biomarkers, the master mix comprising a plurality of PCR primers, the PCR primers comprising oligonucleotide sequences that produce within-type sequences for at least one of genes E6 and E7 of at least one HPV type selected from the group consisting of 16, 18, 31, 33, 45 and 52;
executing a single multiplexed PCR amplification to produce a plurality of amplified end-label amplicons; and
identifying HPV types in the plurality of amplified end-label amplicons using electrophoresis.

2. The method of claim 1 wherein the oligonucleotide sequences are selected from the group consisting of:
Sequence 1
TGGACCGGTCGATGTATGTCTTGT
Sequence 2
TACGCACAACCGAAGCGTAGAGTC
Sequence 3
AGTGTGACTCTACGCTTCGGTTGT
Sequence 4
GTGTGCCCATTAACAGGTCTTCCA
Sequence 5
ACTATAGAGGCCAGTGCCATTCGT
Sequence 6
TCGTCGGGCTGGTAAATGTTGATG
Sequence 7
CATCAACATTTACCAGCCCGACGA
Sequence 8
AAACAGCTGCTGGAATGCTCGAAG
Sequence 9
AACATAGGAGGAAGGTGGACAGGA
Sequence 10
GTGTGCTCTGTACACACAAACGAAG
Sequence 11
GAGGACACAAGCCAACGTTAAAGG
Sequence 12
GGTTCGTAGGTCACTTGCTGTACT
Sequence 13
GACAGTACCGAGGGCAGTGTAATA
Sequence 14
TACTTGTGTTTCCCTACGTCTGCGA
Sequence 15
GCGTGTGTATTATGTGCCTACGCT
Sequence 16
TTACACTTGGGTCACAGGTCGG
Sequence 17
AGGTGACACTATAGAATATGGACCGGTCGATGTATGTCTTGT
Sequence 18
GTACGACTCACTATAGGGATACGCACAACCGAAGCGTAGAGTC
Sequence 19
AGGTGACACTATAGAATAAGTGTGACTCTACGCTTCGGTTGT
Sequence 20
GTACGACTCACTATAGGGAGTGTGCCCATTAACAGGTCTTCCA
Sequence 21
AGGTGACACTATAGAATAACTATAGAGGCCAGTGCCATTCGT
Sequence 22
GTACGACTCACTATAGGGATCGTCGGGCTGGTAAATGTTGATG
Sequence 23
AGGTGACACTATAGAATACATCAACATTTACCAGCCCGACGA
Sequence 24
GTACGACTCACTATAGGGAAAACAGCTGCTGGAATGCTCGAAG
Sequence 25
AGGTGACACTATAGAATAAACATAGGAGGAAGGTGGACAGGA
Sequence 26
GTACGACTCACTATAGGGAGTGTGCTCTGTACACACAAACGAAG
Sequence 27
AGGTGACACTATAGAATAGAGGACACAAGCCAACGTTAAAGG
Sequence 28
GTACGACTCACTATAGGGAGGTTCGTAGGTCACTTGCTGTACT
Sequence 29
AGGTGACACTATAGAATAGACAGTACCGAGGGCAGTGTAATA
Sequence 30
GTACGACTCACTATAGGGATACTTGTGTTTCCCTACGTCTGCGA
Sequence 31
AGGTGACACTATAGAATAGCGTGTGTATTATGTGCCTACGCT
Sequence 32
GTACGACTCACTATAGGGATTACACTTGGGTCACAGGTCGG
and the reverse complements thereof.

3. The method of claim 1 wherein the oligonucleotide sequences are selected from the group consisting of:
Sequence 1
TGGACCGGTCGATGTATGTCTTGT
Sequence 2
TACGCACAACCGAAGCGTAGAGTC
Sequence 3
AGTGTGACTCTACGCTTCGGTTGT
Sequence 4
GTGTGCCCATTAACAGGTCTTCCA
Sequence 5
ACTATAGAGGCCAGTGCCATTCGT
Sequence 6
TCGTCGGGCTGGTAAATGTTGATG
Sequence 7
CATCAACATTTACCAGCCCGACGA
Sequence 8
AAACAGCTGCTGGAATGCTCGAAG
Sequence 9
AACATAGGAGGAAGGTGGACAGGA
Sequence 10
GTGTGCTCTGTACACACAAACGAAG
Sequence 11
GAGGACACAAGCCAACGTTAAAGG
Sequence 12
GGTTCGTAGGTCACTTGCTGTACT
Sequence 13
GACAGTACCGAGGGCAGTGTAATA
Sequence 14
TACTTGTGTTTCCCTACGTCTGCGA
Sequence 15
GCGTGTGTATTATGTGCCTACGCT
Sequence 16
TTACACTTGGGTCACAGGTCGG
and the reverse complements thereof.

4. The method of claim 1 wherein the oligonucleotide sequences are selected from the group consisting of:
Sequence 17
AGGTGACACTATAGAATATGGACCGGTCGATGTATGTCTTGT
Sequence 18
GTACGACTCACTATAGGGATACGCACAACCGAAGCGTAGAGTC
Sequence 19
AGGTGACACTATAGAATAAGTGTGACTCTACGCTTCGGTTGT
Sequence 20
GTACGACTCACTATAGGGAGTGTGCCCATTAACAGGTCTTCCA
Sequence 21
AGGTGACACTATAGAATAACTATAGAGGCCAGTGCCATTCGT
Sequence 22
GTACGACTCACTATAGGGATCGTCGGGCTGGTAAATGTTGATG
Sequence 23
AGGTGACACTATAGAATACATCAACATTTACCAGCCCGACGA
Sequence 24
GTACGACTCACTATAGGGAAAACAGCTGCTGGAATGCTCGAAG
Sequence 25
AGGTGACACTATAGAATAAACATAGGAGGAAGGTGGACAGGA
Sequence 26
GTACGACTCACTATAGGGAGTGTGCTCTGTACACACAAACGAAG
Sequence 27
AGGTGACACTATAGAATAGAGGACACAAGCCAACGTTAAAGG
Sequence 28
GTACGACTCACTATAGGGAGGTTCGTAGGTCACTTGCTGTACT
Sequence 29
AGGTGACACTATAGAATAGACAGTACCGAGGGCAGTGTAATA
Sequence 30
GTACGACTCACTATAGGGATACTTGTGTTTCCCTACGTCTGCGA
Sequence 31
AGGTGACACTATAGAATAGCGTGTGTATTATGTGCCTACGCT
Sequence 32
GTACGACTCACTATAGGGATTACACTTGGGTCACAGGTCGG
and the reverse complements thereof.

5. The method of claim 1 wherein the separating of cells positive for multiple biomarkers comprises separating cells exhibiting over expression of any two proteins selected from the group consisting of p16^{INK4a}, mcm5, PCNA, and any other protein biomarker associated with cervical epithelial cell transformation.

6. The method of claim 3 wherein the electrophoresis is gel electrophoresis.

7. The method of claim 4 wherein the electrophoresis is capillary electrophoresis.

8. The method of claim 1 further comprising thermocycling the amplified end-label amplicons, wherein the thermocycling comprises a plurality of repeated cycles and each cycle comprises a denaturation step and an annealing/extension step.

9. The method of claim 8 wherein the thermocycling comprises less than about 35 cycles.

10. The method of claim 1 wherein the executing a single multiplexed PCR amplification to produce a plurality of amplified end-label amplicons is carried out without a pre-amplification by degenerate HPV primers.

11. The method of claim 1 wherein the fluorescently tagged antibodies, used during cell sorting, are directed toward two or more protein biomarkers known to have functional relationships with transformation, tumor initiation, tumor progression, or tumor invasion, such as, but not limited to, EGFR1, VEGF, HIF-1α, IGFBP-3, P-cadherin, MCM7, p16^{4a} or MCM5.

12. (canceled)

12. (canceled)

13. (canceled)

14. An oligonucleotide sequence that produce within-type sequences for at least one of genes E6 and E7 of at least one HPV type selected from the group consisting of 16, 18, 31, 33, 45 and 52, the oligonucleotide sequence selected from the group consisting of:
Sequence 1
TGGACCGGTCGATGTATGTCTTGT
Sequence 2
TACGCACAACCGAAGCGTAGAGTC
Sequence 3
AGTGTGACTCTACGCTTCGGTTGT
Sequence 4
GTGTGCCCATTAACAGGTCTTCCA
Sequence 5
ACTATAGAGGCCAGTGCCATTCGT
Sequence 6
TCGTCGGGCTGGTAAATGTTGATG
Sequence 7
CATCAACATTTACCAGCCCGACGA
Sequence 8
AAACAGCTGCTGGAATGCTCGAAG
Sequence 9
AACATAGGAGGAAGGTGGACAGGA
Sequence 10
GTGTGCTCTGTACACACAAACGAAG
Sequence 11
GAGGACACAAGCCAACGTTAAAGG
Sequence 12
GGTTCGTAGGTCACTTGCTGTACT
Sequence 13
GACAGTACCGAGGGCAGTGTAATA
Sequence 14
TACTTGTGTTTCCCTACGTCTGCGA
Sequence 15
GCGTGTGTATTATGTGCCTACGCT
Sequence 16
TTACACTTGGGTCACAGGTCGG
Sequence 17
AGGTGACACTATAGAATATGGACCGGTCGATGTATGTCTTGT
Sequence 18
GTACGACTCACTATAGGGATACGCACAACCGAAGCGTAGAGTC
Sequence 19
AGGTGACACTATAGAATAAGTGTGACTCTACGCTTCGGTTGT
Sequence 20
GTACGACTCACTATAGGGAGTGTGCCCATTAACAGGTCTTCCA
Sequence 21
AGGTGACACTATAGAATAACTATAGAGGCCAGTGCCATTCGT
Sequence 22
GTACGACTCACTATAGGGATCGTCGGGCTGGTAAATGTTGATG
Sequence 23
AGGTGACACTATAGAATACATCAACATTTACCAGCCCGACGA
Sequence 24
GTACGACTCACTATAGGGAAAACAGCTGCTGGAATGCTCGAAG
Sequence 25
AGGTGACACTATAGAATAAACATAGGAGGAAGGTGGACAGGA
Sequence 26
GTACGACTCACTATAGGGAGTGTGCTCTGTACACACAAACGAAG
Sequence 27
AGGTGACACTATAGAATAGAGGACACAAGCCAACGTTAAAGG
Sequence 28
GTACGACTCACTATAGGGAGGTTCGTAGGTCACTTGCTGTACT
Sequence 29
AGGTGACACTATAGAATAGACAGTACCGAGGGCAGTGTAATA
Sequence 30
GTACGACTCACTATAGGGATACTTGTGTTTCCCTACGTCTGCGA
Sequence 31
AGGTGACACTATAGAATAGCGTGTGTATTATGTGCCTACGCT
Sequence 32
GTACGACTCACTATAGGGATTACACTTGGGTCACAGGTCGG
and the reverse complements thereof.

15. A PCR primer comprising an oligonucleotide sequence of claim 14.

16. A PCR master mix comprising at least one PCR primer of claim 15.


SEQUENCE LISTING (32 sequences)

SEQ ID NO: 1 - DNA, 24 bp, artificial
  synthetic oligonucleotide primer
tggaccggtcgatgtatgtcttgt

SEQ ID NO: 2 - DNA, 24 bp, artificial
  synthetic oligonucleotide primer
tacgcacaaccgaagcgtagagtc

SEQ ID NO: 3 - DNA, 24 bp, artificial
  synthetic oligonucleotide primer
agtgtgactctacgcttcggttgt

SEQ ID NO: 4 - DNA, 24 bp, artificial
  synthetic oligonucleotide primer
gtgtgcccattaacaggtcttcca

SEQ ID NO: 5 - DNA, 24 bp, artificial
  synthetic oligonucleotide primer
actatagaggccagtgccattcgt

SEQ ID NO: 6 - DNA, 24 bp, artificial
  synthetic oligonucleotide primer
tcgtcgggctggtaaatgttgatg

SEQ ID NO: 7 - DNA, 24 bp, artificial
  synthetic oligonucleotide primer
catcaacatttaccagcccgacga

SEQ ID NO: 8 - DNA, 24 bp, artificial
  synthetic oligonucleotide primer
aaacagctgctggaatgctcgaag

SEQ ID NO: 9 - DNA, 24 bp, artificial
  synthetic oligonucleotide primer
aacataggaggaaggtggacagga

SEQ ID NO: 10 - DNA, 25 bp, artificial
  synthetic oligonucleotide primer
gtgtgctctgtacacacaaacgaag

SEQ ID NO: 11 - DNA, 24 bp, artificial
  synthetic oligonucleotide primer
gaggacacaagccaacgttaaagg

SEQ ID NO: 12 - DNA, 24 bp, artificial
  synthetic oligonucleotide primer
ggttcgtaggtcacttgctgtact

SEQ ID NO: 13 - DNA, 24 bp, artificial
  synthetic oligonucleotide primer
gacagtaccgagggcagtgtaata

SEQ ID NO: 14 - DNA, 25 bp, artificial
  synthetic oligonucleotide primer
tacttgtgtttccctacgtctgcga

SEQ ID NO: 15 - DNA, 24 bp, artificial
  synthetic oligonucleotide primer
gcgtgtgtattatgtgcctacgct

SEQ ID NO: 16 - DNA, 22 bp, artificial
  synthetic oligonucleotide primer
ttacacttgggtcacaggtcgg

SEQ ID NO: 17 - DNA, 42 bp, artificial
  synthetic oligonucleotide primer
aggtgacactatagaatatggaccggtcgatgtatgtcttgt

SEQ ID NO: 18 - DNA, 43 bp, artificial
  synthetic oligonucleotide primer
gtacgactcactatagggatacgcacaaccgaagcgtagagtc

SEQ ID NO: 19 - DNA, 42 bp, artificial
  synthetic oligonucleotide primer
aggtgacactatagaataagtgtgactctacgcttcggttgt

SEQ ID NO: 20 - DNA, 43 bp, artificial
  synthetic oligonucleotide primer
gtacgactcactatagggagtgtgcccattaacaggtcttcca

SEQ ID NO: 21 - DNA, 42 bp, artificial
  synthetic oligonucleotide primer
aggtgacactatagaataactatagaggccagtgccattcgt

SEQ ID NO: 22 - DNA, 43 bp, artificial
  synthetic oligonucleotide primer
gtacgactcactatagggatcgtcgggctggtaaatgttgatg

SEQ ID NO: 23 - DNA, 42 bp, artificial
  synthetic oligonucleotide primer
aggtgacactatagaatacatcaacatttaccagcccgacga

SEQ ID NO: 24 - DNA, 43 bp, artificial
  synthetic oligonucleotide primer
gtacgactcactatagggaaaacagctgctggaatgctcgaag

SEQ ID NO: 25 - DNA, 42 bp, artificial
  synthetic oligonucleotide primer
aggtgacactatagaataaacataggaggaaggtggacagga

SEQ ID NO: 26 - DNA, 44 bp, artificial
  synthetic oligonucleotide primer
gtacgactcactatagggagtgtgctctgtacacacaaacgaag

SEQ ID NO: 27 - DNA, 42 bp, artificial
  synthetic oligonucleotide primer
aggtgacactatagaatagaggacacaagccaacgttaaagg

SEQ ID NO: 28 - DNA, 43 bp, artificial
  synthetic oligonucleotide primer
gtacgactcactatagggaggttcgtaggtcacttgctgtact

SEQ ID NO: 29 - DNA, 42 bp, artificial
  synthetic oligonucleotide primer
aggtgacactatagaatagacagtaccgagggcagtgtaata

SEQ ID NO: 30 - DNA, 44 bp, artificial
  synthetic oligonucleotide primer
gtacgactcactatagggatacttgtgtttccctacgtctgcga

SEQ ID NO: 31 - DNA, 42 bp, artificial
  synthetic oligonucleotide primer
aggtgacactatagaatagcgtgtgtattatgtgcctacgct

SEQ ID NO: 32 - DNA, 41 bp, artificial
  synthetic oligonucleotide primer
gtacgactcactatagggattacacttgggtcacaggtcgg